(19) United States
(12) Patent Application Publication
(10) Pub. No.: US 2019/0343718 A1
(43) Pub. Date: Nov. 14, 2019

(54) PILLOW

(71) Applicants: Hye Jeong SHIN, Namyangju-si (KR); Ji Woong PARK, Namyangju-si (KR)

(72) Inventors: Hye Jeong SHIN, Namyangju-si (KR); Ji Woong PARK, Namyangju-si (KR)

(21) Appl. No.: 16/476,170

(22) PCT Filed: Jan. 4, 2018
(86) PCT No.: PCT/KR2018/000161

§ 371 Date: Jul. 5, 2019

(30) Foreign Application Priority Data
Jan. 5, 2017 (KR) 10-2017-0001939

(51) Int. Cl. A61H 39/04; A47G 9/10; A61F 7/00; A61H 23/02

(52) U.S. Cl. CPC A61H 39/04; A47G 9/1045; A47G 9/1009; A61F 7/007; A61H 2201/1607; A61F 2007/0002; A61F 2007/0088; A61H 2205/02; A61H 2201/1611; A61H 23/02

(57) ABSTRACT

The pillow of the present disclosure comprises: a first unit body and a second unit body which are arranged side by side in the left and right directions and are connected to each other with a variable gap therebetween; a third unit body which is coupled to the first unit body so as to move relative thereto in the forward and backward directions; a fourth unit body which is coupled to the second unit body so as to move relative thereto in the forward and backward directions, disposed in parallel to the third unit body in the left and right directions and coupled thereto with a variable gap therebetween; a first gap adjusting unit for allowing the third unit body and the fourth unit body to get further apart from or closer to each other in the forward and backward directions with respect to the first unit body and the second unit body; and a second gap adjusting unit for allowing the first unit body and the third unit body to get further apart from or closer to each other in the left and right directions with respect to the second unit body and the fourth unit body.

### TECHNICAL FIELD

The present invention relates to a pillow, and more particularly, to a pillow conveniently supporting a user's neck.

### BACKGROUND ART

Pillows are bedding that support the user's head when sleeping or resting. In the case of existing pillows, a pillow insert serving as a cushion is inserted into a pillow body.

However, such existing pillows merely serve to support the head, but do not have other functions. Accordingly, existing pillows are not sufficient to meet diverse needs of consumers requiring various functions. To address such a problem, a multipurpose pillow manufactured according to the shape of a human body has recently been proposed. However, such a pillow has a limitation in that functions and effects thereof are reduced or are not normally exhibited when the shape and size of a user's head are different from standard shapes and size. Meanwhile, some existing pillows are provided with protrusions for acupressure. However, since such protrusions are arranged at certain intervals, some users may be inconvenient to use or it may be difficult to exhibit appropriate acupressure effect, depending upon the user's body shape or head shape.

Further, since existing pillows are laid flat on a floor, it may be difficult to adjust the tilt thereof to fit a user's body and thus conveniently use the same.

### DISCLOSURE

### Technical Problem

Therefore, the present invention has been made in view of the above problems, and it is one object of the present invention to provide a pillow that is adjustable to fit a user's body.

### Technical Solution

In accordance with one aspect of the present invention, provided is a pillow including a first unit body and a second unit body configured to be arranged side by side in left and right directions and coupled to each other such that an interval therebetween is adjustable; a third unit body coupled to the first unit body so as to move relative thereto in the forward and backward directions; a fourth unit body coupled to the second unit body so as to move relative thereto in the forward and backward directions, disposed in parallel to the third unit body in left and right directions, and coupled to the third unit body such that an interval therebetween is adjustable; a first gap adjustment unit allowing the third unit body and the fourth unit body to move away from or approach the first unit body and the second unit body in forward and backward directions; and a second gap adjustment unit allowing the first unit body and the third unit body to move away from or approach the second unit body and the fourth unit body in left and right directions.

Meanwhile, the first gap adjustment unit may include a first rotation shaft disposed in left and right directions inside the first unit body; a first operation member configured to be exposed to the outside such that a user can operate outside, and coupled to the first rotation shaft; a second rotation shaft disposed to cross the first unit body and the third unit body in forward and backward directions, screw connected to the third unit body, and configured to rotate in conjunction with rotation of the first rotation shaft so that the third unit body moves away from or approach the first unit body; and a third rotation shaft disposed to cross the second unit body and the fourth unit body in forward and backward directions and screw connected to the fourth unit body.

Meanwhile, the second gap adjustment unit may include a fourth rotation shaft disposed to cross the first unit body and the second unit body in left and right directions, and screw connected to the first unit body; a second rotation shaft exposed to the outside such that a user operates outside, and coupled to the fourth rotation shaft; and a fifth rotation shaft disposed to cross the third unit body and the fourth unit body in left and right directions and screw connected to the third unit body.

Meanwhile, the pillow may include a height adjustment unit allowing the first unit body, the second unit body, and the third unit body, and the fourth unit body to respectively move away from or approach a floor.

Meanwhile, the height adjustment unit may include a sixth rotation shaft configured to connect the first unit body and the second unit body to each other, a length of the sixth rotation shaft being adjustable; a seventh rotation shaft configured to connect the third unit body and the fourth unit body to each other, a length of the seventh rotation shaft being adjustable; third operation members exposed to the outside such that a user can operate outside, and respectively coupled to the sixth rotation shaft and the seventh rotation shaft; pinion gears disposed inside each of the first unit body, the second unit body, the third unit body, and the fourth unit body, and coupled to each of the sixth rotation shaft and the seventh rotation shaft; rack gears disposed at each of the first unit body, the second unit body, the third unit body, and the fourth unit body to move in up and down directions and to be engaged with the pinion gears; and support members formed at ends of the rack gears.

Meanwhile, the pillow may include first acupressure protrusions formed on portions, which are in contact with a user's neck, of the third unit body and the fourth unit body; second acupressure protrusions on portions, which are in contact with a user's neck, of the first unit body and the second unit body; and third acupressure protrusions configured to have a relatively smaller height than a height of the first acupressure protrusions, and coupled with portions, which are in contact with a user's neck, of the third unit body and the fourth unit body to be capable of moving in a linear reciprocating manner in up and down directions.

Meanwhile, the pillow may include a power generation part configured to generate power such that the third acupressure protrusions are moved; a power supply part electrically connected to the power generation part to supply power to the power generation part; a controller electrically connected to the power generation part to control operation of the power generation part; and an operation button formed at an outer surface of at least one of the first unit body, the second unit body, the third unit body, and the fourth unit body, and electrically connected to the controller to generate an operation signal for controlling the power generation part by the controller.

Meanwhile, the pillow may further include a heating coil disposed inside at least one of the first unit body, the second unit body, the third unit body, and the fourth unit body to generate heat.

Meanwhile, the pillow may further include a vibration generation unit installed inside at least one of the first unit body, the second unit body, the third unit body, and the fourth unit body to generate vibration.

[Advantageous effects] As apparent from the fore-going, the present invention advantageously provides a pillow, an overall size of which is adjustable to correspond to the size of the user's body and, accordingly, which can provide a healthy sleep and comfort to the user by ideally supporting the back of the head, i.e., the head and the neck, of the user when sleeping. Additionally, the pillow according to the present invention can help treat neck disc patients.

In addition, the height of the pillow according to the present invention is adjustable, thereby facilitating a good night's sleep in a comfortable position. Further, the tilt of the pillow is adjustable, thereby making a user feel more comfortable.

### DESCRIPTION OF DRAWINGS

FIG. 1 illustrates a perspective view of a pillow according to an embodiment of the present invention.

FIG. 2a illustrates an enlarged view of a first gap adjustment unit inside the pillow of FIG. 1.

FIG. 2b is a view illustrating a state in which the size of a pillow is increased in forward and backward directions by a first gap adjustment unit.

FIG. 3a illustrates an enlarged view of a second gap adjustment unit inside the pillow of FIG. 1.

FIG. 3b is a view illustrating a state in which the size of a pillow is increased in left and right directions by a second gap adjustment unit.

FIG. 4 illustrates a cross-sectional view taken along line IV-IV′ of the pillow of FIG. 2a provided with a height adjustment unit.

FIG. 5 is a view illustrating a state in which a pillow is inclined relative to the ground.

FIG. 6 is a view schematically illustrating a detailed configuration capable of driving third acupressure protrusions of the pillow according to the present invention.

### BEST MODE FOR CARRYING OUT THE INVENTION

As an exemplary embodiment of the present invention, provided is a pillow including a first unit body and a second unit body configured to be arranged side by side in left and right directions and coupled to each other such that an interval therebetween is adjustable; a third unit body coupled to the first unit body so as to move relative thereto in the forward and backward directions; a fourth unit body coupled to the second unit body so as to move relative thereto in the forward and backward directions, disposed in parallel to the third unit body in left and right directions, and coupled to the third unit body such that an interval therebetween is adjustable; a first gap adjustment unit allowing the third unit body and the fourth unit body to move away from or approach the first unit body and the second unit body in forward and backward directions; and a second gap adjustment unit allowing the first unit body and the third unit body to move away from or approach the second unit body and the fourth unit body in left and right directions.

### MODE FOR CARRYING OUT THE INVENTION

Exemplary embodiments of the present invention are described in detail so as for those of ordinary skill in the art to easily implement with reference to the accompanying drawings. However, the present invention may be implemented in various different forms and is not limited to these embodiments.

To clearly describe the present invention, a part without concerning to the description is omitted in the drawings, and like reference numerals in the specification denote like elements.

In various embodiments, the elements having the same construction are representatively described in a first embodiment by using the same reference numeral, and constructions different from that described in the first embodiment are described in the other embodiments.

In the specification, when it is described that a certain part is “connected” to another part, it should be understood that the certain part may be directly connected to another part or electrically connected to another part via another part in the middle. In addition, when a certain part “includes” a certain component, this indicates that the part may further include another component instead of excluding another component unless there is no different disclosure.

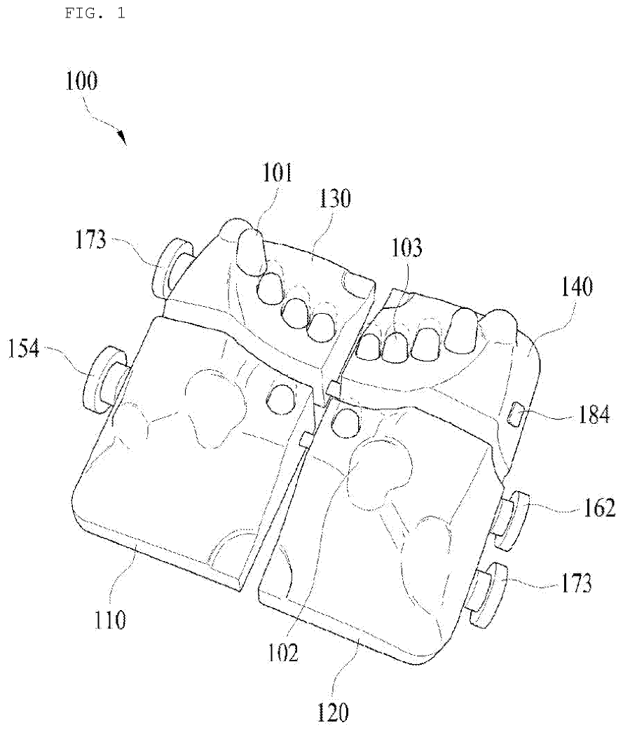

FIG. 1 illustrates a perspective view of a pillow according to an embodiment of the present invention.

Referring to FIG. 1, a pillow 100 according to an embodiment of the present invention includes a first unit body 110, a second unit body 120, a third unit body 130, a fourth unit body 140, a first gap adjustment unit 150, and a second gap adjustment unit 160.

The first unit body 110 and the second unit body 120 are arranged side by side in left and right directions, and are coupled to each other such that an interval therebetween is adjustable. The first unit body 110 and the second unit body 120 may be symmetrical with respect to each other on a plane, but the present invention is not limited thereto.

The third unit body 130 is coupled to the first unit body 110 so as to move relative thereto in the forward and backward directions.

The fourth unit body 140 is coupled to the second unit body 120 so as to move relative thereto in the forward and backward directions. The fourth unit body 140 and the third unit body 130 are arranged side by side in left and right directions, and are coupled to each other such that an interval therebetween is adjustable. The fourth unit body 140 and the third unit body 130 may be symmetrical with respect to each other on a plane, but the present invention is not limited thereto.

Skeletons of the first unit body 110, the second unit body 120, the third unit body 130, and the fourth unit body 140 may be formed of a rigid material, and the skeletons may be wrapped with a soft material. Accordingly, a user may feel soft upon use of the pillow 100 according to an embodiment of the present invention.

Although not shown in the drawings, the first unit body 110, the second unit body 120, the third unit body 130, and the fourth unit body 140 may be wrapped with a separate cover member (not shown). The cover member may prevent the first unit body 110, the second unit body 120, the third unit body 130, and the fourth unit body 140 from being contaminated with external foreign substances. A material of the cover member may be formed of, for example, leather, artificial leather, synthetic resin, etc., but the present invention is not limited thereto.

The first gap adjustment unit 150 allows the third unit body 130 and the fourth unit body 140 to move away from or approach the first unit body 110 and the second unit body 120 in forward and backward directions.

The second gap adjustment unit 160 allows the first unit body 110 and the third unit body 130 to move away from or approach the second unit body 120 and the fourth unit body 140 in left and right directions. Embodiments of detailed structures of the second gap adjustment unit 160 and the first gap adjustment unit 150 are described below.

Front-to-back intervals and left-to-right intervals of the first unit body 110, the second unit body 120, the third unit body 130, and the fourth unit body 140 of the pillow 100 according to an embodiment of the present invention may be adjusted by the first gap adjustment unit 150 and the second gap adjustment unit 160. That is, since an overall size of the pillow 100 according to an embodiment of the present invention can be adjusted, the pillow 100 can provide a healthy sleep and comfort to a user by ideally supporting the back of the head, i.e., the head and the neck, of the user when sleeping. Additionally, the pillow 100 according to the present invention can help treat neck disc patients.

Hereinafter, an embodiment of a structure of the pillow 100 according to an embodiment of the present invention is described in detail.

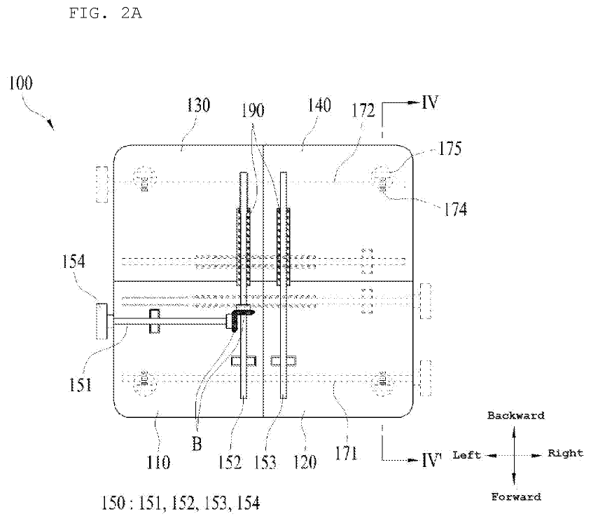

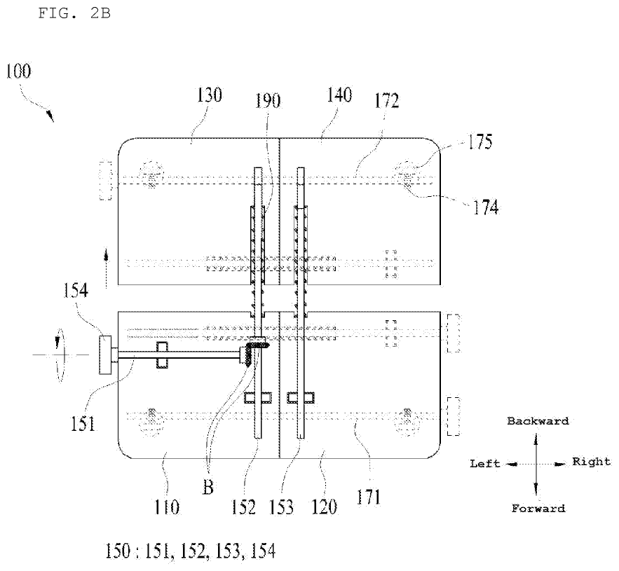

FIG. 2a illustrates an enlarged view of a first gap adjustment unit inside the pillow of FIG. 1, and FIG. 2b is a view illustrating a state in which the size of a pillow is increased in forward and backward directions by a first gap adjustment unit.

Referring to FIGS. 2a and 2b, the first gap adjustment unit 150 may include, for example, a first rotation shaft 151, a first operation member 154, a second rotation shaft 152, and a third rotation shaft 153.

The first rotation shaft 151 is disposed in left and right directions inside the first unit body 110.

The first operation member 154 may be exposed to the outside such that a user can operate the same outside, and may be coupled with the first rotation shaft 151. For example, the first operation member 154 may be rotatably coupled with the first unit body 110.

Here, the first operation member 154 and the first rotation shaft 151 may be connected to each other by a separate motor (not shown). That is, when a user operates (rotates or presses) the first operation member 154, the first rotation shaft 151 may be rotated while the motor is operated.

The second rotation shaft 152 may be disposed to cross the first unit body 110 and the third unit body 130 in forward and backward directions. The second rotation shaft 152 may be disposed to be orthogonal to the first rotation shaft 151. The second rotation shaft 152 is screw connected to the third unit body 130 and rotates in conjunction with rotation of the first rotation shaft 151, so that the third unit body 130 moves away from or approaches the first unit body 110.

To accomplish this, the second rotation shaft 152 and the first rotation shaft 151 may be connected to each other by two bevel gears (B) so that the rotational force of the first rotation shaft 151 is transmitted to the second rotation shaft 152. Alternatively, the second rotation shaft 152 and the first rotation shaft 151 may be connected to each other by a worm and a worm gear.

The third rotation shaft 153 may be disposed to cross the second unit body 120 and the fourth unit body 140 in forward and backward directions. The third rotation shaft 153 may guide the second unit body 120 and the fourth unit body 140 to stably move relative to each other. The third rotation shaft 153 may be a length-adjustable spline.

The first gap adjustment unit 150 allows the third unit body 130 and the fourth unit body 140 to move relative to the first unit body 110 and the second unit body 120 in forward and backward directions. For example, when a user rotates the first operation member 154 clockwise, the third unit body 130 and the fourth unit body 140 may be moved away from the first unit body 110 and the second unit body 120. On the contrary, when a user rotates the first operation member 154 counterclockwise, the third unit body 130 and the fourth unit body 140 may approach the first unit body 110 and the second unit body 120.

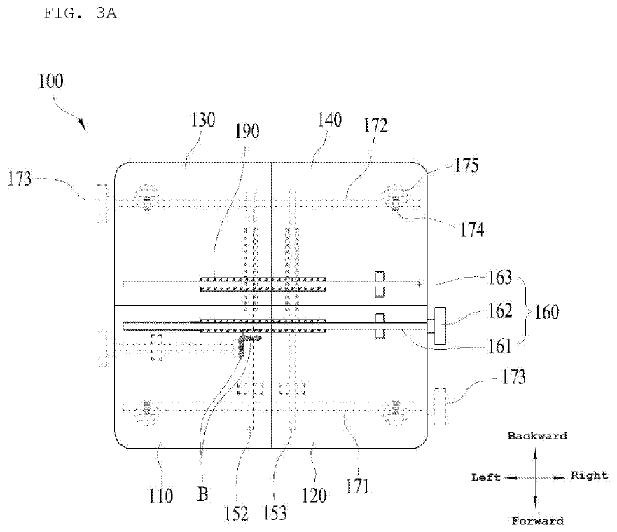

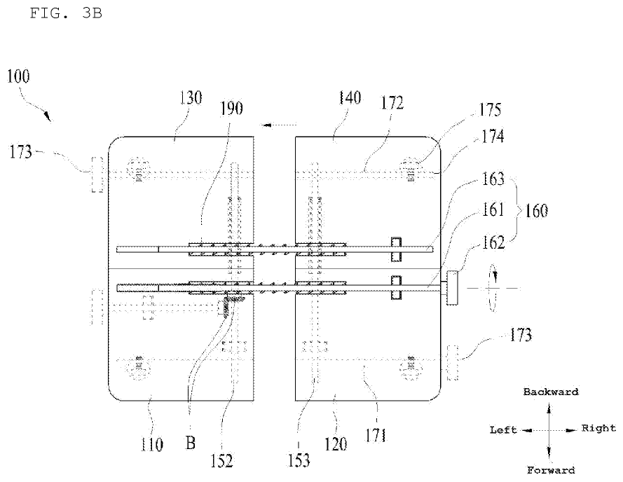

FIG. 3a illustrates an enlarged view of a second gap adjustment unit inside the pillow of FIG. 1, and FIG. 3b is a view illustrating a state in which the size of a pillow is increased in left and right directions by a second gap adjustment unit.

Referring to FIGS. 3a and 3b, the second gap adjustment unit 160 may include, for example, a fourth rotation shaft 161, a second rotation shaft 162, and a fifth rotation shaft 163.

The fourth rotation shaft 161 may be disposed to cross the first unit body 110 and the second unit body 120 in left and right directions. The fourth rotation shaft 161 may be screw connected to the first unit body 110.

The second rotation shaft 162 may be exposed to the outside such that a user can operate the same outside, and may be coupled with the fourth rotation shaft 161. For example, the second rotation shaft 162 may be rotatably coupled with the second unit body 120. The shape of the second rotation shaft 162 may be similar to that of the first operation member 154.

Here, the second rotation shaft 162 and the fourth rotation shaft 161 may be connected to each other by a separate motor (not shown). That is, when a user operates (rotates or presses) the second rotation shaft 162, the fourth rotation shaft 161 may be rotated while the motor is operated.

When a user rotates the second rotation shaft 162 in one direction, the first unit body 110 may move along the fourth rotation shaft 161. Here, since the fourth rotation shaft 161 is screw connected to the first unit body 110, the first unit body 110 may approach or move away from the second unit body 120 according to a rotation direction of the fourth rotation shaft 161. Here, since the first unit body 110 is connected to the third unit body 130 by the second rotation shaft 152, the first unit body 110 may move along with the third unit body 130.

The fifth rotation shaft 163 may be disposed to cross the third unit body 130 and the fourth unit body 140 in left and right directions. The fifth rotation shaft 163 may guide the third unit body 130 and the fourth unit body 140 to stably move relative to each other. The fifth rotation shaft 163 may be a length-adjustable spline.

Meanwhile, the pillow 100 according to an embodiment of the present invention may further include a plurality of elastic members 190.

The elastic members 190 may be disposed to respectively wrap the second rotation shaft 152, the third rotation shaft 153, the fourth rotation shaft 161, and the fifth rotation shaft 163. In addition, the elastic members 190 may be interposed between the unit bodies. More particularly, the elastic members 190 may be interposed between the first unit body 110 and the second unit body 120. The elastic members 190 may be interposed between the first unit body 110 and the third unit body 130. The elastic members 190 may be interposed between the third unit body 130 and the fourth unit body 140. The elastic members 190 may be interposed between the second unit body 120 and the fourth unit body 140.

The first unit body 110, the second unit body 120, the third unit body 130, and the fourth unit body 140 can stably maintain corresponding positions thereof in a state spaced from each other by elastic force of the elastic members 190. For this, the elastic members 190 may be, for example, compression springs.

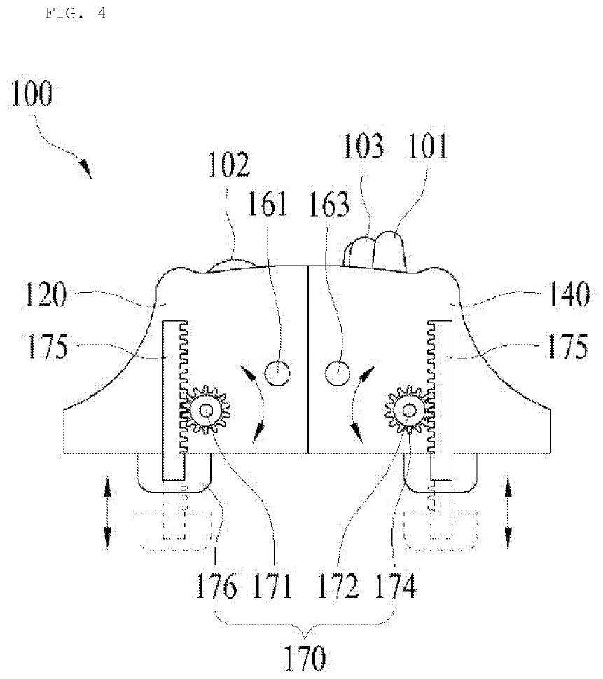

FIG. 4 illustrates a cross-sectional view taken along line IV-IV′ of the pillow of FIG. 2a provided with a height adjustment unit.

Referring to FIG. 4, the pillow 100 according to an embodiment of the present invention may include a height adjustment unit 170.

The height adjustment unit 170 allows the first unit body 110, the second unit body 120, the third unit body 130, and the fourth unit body 140 to respectively move away from or approach a floor.

The height adjustment unit 170 may include, for example, a sixth rotation shaft 171, a seventh rotation shaft 172, third operation members 173, pinion gears 174, rack gears 175, and support members 176.

The sixth rotation shaft 171 may connect the first unit body 110 and the second unit body 120 to each other, and the length of the sixth rotation shaft 171 may be adjustable.

The seventh rotation shaft 172 may connect the third unit body 130 and the fourth unit body 140 to each other, and the length of the seventh rotation shaft 172 may be adjustable. The structure of the seventh rotation shaft 172 may be similar to that of the sixth rotation shaft 171.

The third operation members 173 may be exposed to the outside such that a user can operate the same outside and may be respectively coupled with the sixth rotation shaft 171 and the seventh rotation shaft 172. That is, one of the two third operation members 173 may be coupled with the sixth rotation shaft 171, and the other one thereof may be coupled to the seventh rotation shaft 172. When a user rotates the third operation members 173, the sixth rotation shaft 171 may be rotated, or the seventh rotation shaft 172 may be rotated.

Here, the third operation members 173 may be respectively connected to the sixth rotation shaft 171 and the seventh rotation shaft 172 by separate motors (not shown). That is, when a user operates (rotates or presses) the third operation members 173, the sixth rotation shaft 171 or the seventh rotation shaft 172 may be rotated while the motor is operated.

Each of the pinion gears 174 is disposed inside each of the first unit body 110, the second unit body 120, the third unit body 130, and the fourth unit body 140, and is coupled to each of the sixth rotation shaft 171 and the seventh rotation shaft 172. That is, four pinion gears 174 may be provided. The pinion gears 174 may be respectively disposed inside the first unit body 110, the second unit body 120, the third unit body 130, and the fourth unit body 140. Two of the pinion gears 174 may be coupled to the sixth rotation shaft 171, and the remaining two pinion gears 174 may be coupled to the seventh rotation shaft 172. The pinion gears 174 may rotate in conjunction with rotation of the sixth rotation shaft 171 and the seventh rotation shaft 172.

The rack gears 175 are disposed at each of the first unit body 110, the second unit body 120, the third unit body 130, and the fourth unit body 140 to move in up and down directions and to be engaged with the rack gears 174. The rack gears 175 may vertically move along a rotation direction of the rack gears 174.

Each of the support members 176 is formed at an end of each of the rack gears 175. The support members 176 may be in contact with the ground. A material of the support members 176 may be, for example, rubber or silicone. Accordingly, it is possible to prevent the pillow 100 according to an embodiment of the present invention from being moved by external force in a state of being placed on a floor or a bed.

In addition, edges of portions, which are in contact with a floor surface, of the support members 176 may be rounded. In the case of the pillow 100 according to an embodiment of the present invention, the heights of the first unit body 110 and the second unit body 120 and the heights of the third unit body 130 and the fourth unit body 140 may be separately adjusted.

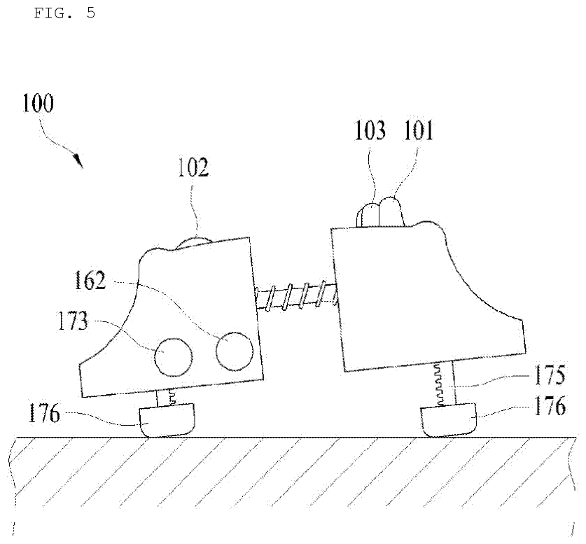

Accordingly, when the height of the first unit body 110 and the second unit body 120 from a floor is different from the height of the third unit body 130 and the fourth unit body 140 from a floor as shown in FIG. 5, the pillow 100 according to an embodiment of the present invention may be placed to be inclined with respect to a floor. Therefore, a user can feel more comfortable when using the pillow 100.

The pillow 100 according to an embodiment of the present invention may include first acupressure protrusions 101, second acupressure protrusions 102, and third acupressure protrusions 103.

The first acupressure protrusions 101 are formed on portions, which are in contact with the user's neck, of the third unit body 130 and the fourth unit body 140. For example, the first acupressure protrusions 101 may be disposed to be adjacent to edges of the third unit body 130 and the fourth unit body 140.

The second acupressure protrusions 102 are formed on portions, which are in contact with the user's neck, of the first unit body 110 and the second unit body 120.

The third acupressure protrusions 103 have a relatively smaller height than that of the first acupressure protrusions 101, and are coupled with portions, which are in contact with the user's neck, of the third unit body 130 and the fourth unit body 140 to be capable of moving in a linear reciprocating manner in up and down directions. One or a plurality of third acupressure protrusions 103 may be provided. When a plurality of third acupressure protrusions 103 is provided, the third acupressure protrusions 103 may be spaced from each other at regular intervals, or intervals between the third acupressure protrusions 103 may be irregular.

Hereinafter, the structure of the pillow 100 according to an embodiment of the present invention allowing the operation of the third acupressure protrusions 103 are described in detail.

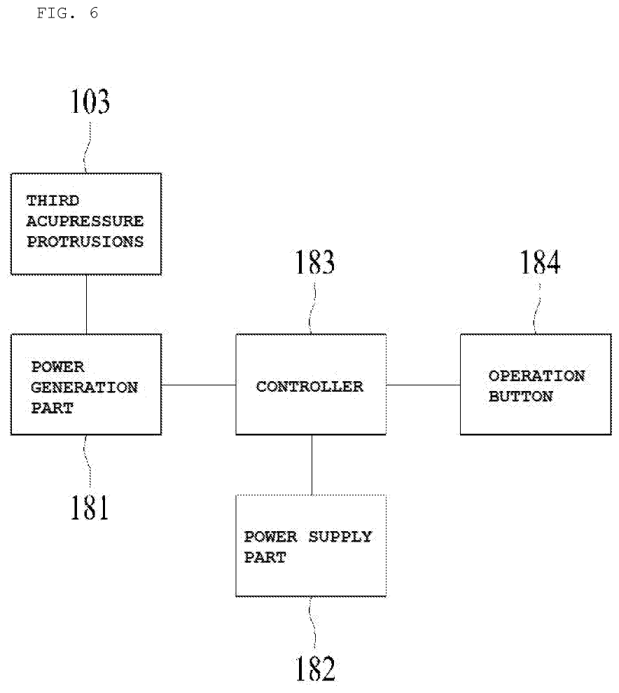

FIG. 6 is a view schematically illustrating a detailed configuration capable of driving third acupressure protrusions of the pillow according to the present invention.

Referring to FIG. 6, the pillow 100 according to an embodiment of the present invention may include a power generation part 181, a power supply part 182, a controller 183, and an operation button 184.

The power generation part 181 generates power such that the third acupressure protrusions 103 are moved. The power generation part 181 may be, for example, a solenoid, but the present invention is not limited thereto. As the power generation part 181, various devices such as a linear motor, a hydraulic cylinder, and a pneumatic cylinder may be selectively used.

The power supply part 182 may be electrically connected to the power generation part 181 to supply power to the power generation part 181. The power supply part 182 may be, for example, a chargeable secondary battery. The power supply part 182 may be, for example, a disposable battery. Alternatively, the power supply part 182 may be, for example, a wall power supply part.

The controller 183 is electrically connected to the power generation part 181 to control operation of the power generation part 181. When a user uses the pillow 100 of the present invention, the controller 183 may serve to control the third acupressure protrusions 103 to stimulate the user's body every specific timing over time. For this, the controller 183 may include programs related to time-dependent operation signals of the power generation part 181. For this, the controller 183 may include a storage in which programs are stored. The storage may be, for example, read only memory (ROM).

The operation button 184 may be formed at an outer surface of at least one of the first unit body 110, the second unit body 120, the third unit body 130, and the fourth unit body 140. The operation button 184 is electrically connected to the controller 183 to generate an operation signal for controlling the power generation part 181 by the controller 183. A user may press the operation button 184 to operate the third acupressure protrusions 103.

The pillow 100 according to an embodiment of the present invention may further include a heating coil (not shown).

The heating coil may be disposed inside at least one of the first unit body 110, the second unit body 120, the third unit body 130, and the fourth unit body 140 to generate heat. The heating coil may receive power from the aforementioned power supply part 182.

A temperature sensor (not shown) may be installed, for example, on one side of the pillow 100 to automatically turn the heating coil on or off according to ambient temperature. For example, a reference temperature of the temperature sensor may be set to 19° C. such that the temperature sensor operates when the ambient temperature is below 19° C., but the present invention is not limited thereto.

When a user uses the pillow 100 according to an embodiment of the present invention in a cold environment, the pillow 100 may be used in a warm state by heat generated by the heating coil.

The pillow 100 according to an embodiment of the present invention may further include a vibration generation unit (not shown).

The vibration generation unit is installed inside at least one of the first unit body 110 and the second unit body 120 and the third unit body 130 and the fourth unit body 140 to generate vibration.

A separate button which is used by a user to operate the vibration generation unit may be installed at an outer surface of the pillow 100. Alternatively, a wireless communication module, such as a Bluetooth module, may be installed inside the pillow 100 to control the operation of the vibration generation unit with a smartphone.

A user may operate the vibration generation unit to massage the neck and relax the neck muscles with the pillow 100 according to an embodiment of the present invention.

As described above, an overall size of the pillow 100 according to the present invention is adjustable to correspond to the size of the user's body. Accordingly, the pillow 100 can provide a healthy sleep and comfort to the user by ideally supporting the back of the head, i.e., the head and the neck, of the user when sleeping. Additionally, the pillow 100 can help treat neck disc patients.

In addition, the height of the pillow 100 is adjustable, thereby facilitating a good night's sleep in a comfortable position. Further, the tilt of the pillow 100 is adjustable, thereby making a user feel more comfortable.

While the present invention has been particularly shown and described with reference to exemplary embodiments thereof, it will be understood by those of ordinary skill in the art that the embodiments are only provided for illustrative purpose and are not intended to limit the scope of the inventive concept defined in the claims. Therefore, it will be understood by those of ordinary skill in the art that various changes in form and details may be made therein without departing from the spirit and scope of the present invention as defined by the following claims. Thus, the technical scope of the present invention should be defined by the technical sprit of the following claims.

1. A pillow, comprising:
a first unit body and a second unit body configured to be arranged side by side in left and right directions and coupled to each other such that an interval therebetween is adjustable;
a third unit body coupled to the first unit body so as to move relative thereto in the forward and backward directions;
a fourth unit body coupled to the second unit body so as to move relative thereto in the forward and backward directions, disposed in parallel to the third unit body in left and right directions, and coupled to the third unit body such that an interval therebetween is adjustable;
a first gap adjustment unit allowing the third unit body and the fourth unit body to move away from or approach the first unit body and the second unit body in forward and backward directions; and
a second gap adjustment unit allowing the first unit body and the third unit body to move away from or approach the second unit body and the fourth unit body in left and right directions.

2. The pillow according to claim 1, wherein the first gap adjustment unit comprises:
a first rotation shaft disposed in left and right directions inside the first unit body;
a first operation member configured to be exposed to the outside such that a user can operate outside, and coupled to the first rotation shaft;
a second rotation shaft disposed to cross the first unit body and the third unit body in forward and backward directions, screw connected to the third unit body, and configured to rotate in conjunction with rotation of the first rotation shaft so that the third unit body moves away from or approach the first unit body; and
a third rotation shaft disposed to cross the second unit body and the fourth unit body in forward and backward directions.

3. The pillow according to claim 1, wherein the second gap adjustment unit comprises:
a fourth rotation shaft disposed to cross the first unit body and the second unit body in left and right directions, and screw connected to the first unit body;
a second rotation shaft exposed to the outside such that a user operates outside, and coupled to the fourth rotation shaft; and
a fifth rotation shaft disposed to cross the third unit body and the fourth unit body in left and right directions.

4. The pillow according to claim 1, comprising a height adjustment unit allowing the first unit body, the second unit body, and the third unit body, and the fourth unit body to respectively move away from or approach a floor.

5. The pillow according to claim 1, wherein the height adjustment unit comprises:
a sixth rotation shaft configured to connect the first unit body and the second unit body to each other, a length of the sixth rotation shaft being adjustable;
a seventh rotation shaft configured to connect the third unit body and the fourth unit body to each other, a length of the seventh rotation shaft being adjustable;
third operation members exposed to the outside such that a user can operate outside, and respectively coupled to the sixth rotation shaft and the seventh rotation shaft;
pinion gears disposed inside each of the first unit body, the second unit body, the third unit body, and the fourth unit body, and coupled to each of the sixth rotation shaft and the seventh rotation shaft;
rack gears disposed at each of the first unit body, the second unit body, the third unit body, and the fourth unit body to move in up and down directions and to be engaged with the pinion gears; and
support members formed at ends of the rack gears.

6. The pillow according to claim 1, comprising:
first acupressure protrusions formed on portions, which are in contact with a user's neck, of the third unit body and the fourth unit body;
second acupressure protrusions on portions, which are in contact with a user's neck, of the first unit body and the second unit body; and
third acupressure protrusions configured to have a relatively smaller height than a height of the first acupressure protrusions, and coupled with portions, which are in contact with a user's neck, of the third unit body and the fourth unit body to be capable of moving in a linear reciprocating manner in up and down directions.

7. The pillow according to claim 6, comprising:
a power generation part configured to generate power such that the third acupressure protrusions are moved;
a power supply part electrically connected to the power generation part to supply power to the power generation part;
a controller electrically connected to the power generation part to control operation of the power generation part; and
an operation button formed at an outer surface of at least one of the first unit body, the second unit body, the third unit body, and the fourth unit body, and electrically connected to the controller to generate an operation signal for controlling the power generation part by the controller.

8. The pillow according to claim 1, further comprising a heating coil disposed inside at least one of the first unit body, the second unit body, the third unit body, and the fourth unit body to generate heat.

9. The pillow according to claim 1, further comprising a vibration generation unit installed inside at least one of the first unit body, the second unit body, the third unit body, and the fourth unit body to generate vibration.
